(19) United States
(12) Patent Application Publication
(10) Pub. No.: US 2005/0171534 A1
(43) Pub. Date: Aug. 4, 2005

(54) Surgical resection device

(75) Inventor: Nagy Habib, London (GB)

Correspondence Address: WILMER CUTLER PICKERING HALE AND DORR LLP, 60 STATE STREET, BOSTON, MA, 02109, US

(73) Assignee: Emcision Limited, London (GB)

(21) Appl. No.: 10/953,989

(22) Filed: Sep. 29, 2004

(30) Foreign Application Priority Data
Sep. 29, 2003 (GB) GB 0322766.7

(51) Int. Cl. A61B 18/14

(52) U.S. Cl. 606/48

(57) ABSTRACT

A surgical organ resection device comprising a plurality of elongate electrodes for insertion into organ tissue, the electrodes being capable of operating in a bipolar manner, and an input for receiving a drive signal for driving the electrode, the elongate electrodes being arranged in a two-dimensional array and/or the device being arranged such that, in use, subsets of the elongate electrodes are driven in turn.

### FIELD OF THE INVENTION

This invention relates to a surgical resection device and in particular to a bipolar resection device. The surgical operation of resectioning refers to the division of one piece of tissue from another piece of tissue.

### BACKGROUND OF THE INVENTION

Hepatic carcinoma (HCC) or liver cancer is a significant cause of death in the developed world. Each year over 18,000 new primary liver tumours are diagnosed in the US. In addition liver secondary tumours are frequently caused by colorectal cancer. Surgical removal of the tumour and surrounding liver tissue is the treatment of choice and, at the current time, liver resection is considered to be the only potentially curative treatment for primary and metastatic liver tumours. The procedure has proven benefit for patients with colorectal liver tumours.

A major problem facing hepatic surgeons is the extent to which the liver bleeds when it is cut. As well as making the surgeon's task difficult by obscuring vision, blood loss during liver surgery is a well recognised and widely documented cause of morbidity and mortality. A patient undergoing the resection of a liver tumour may lose two to three units of blood and in some cases as much as 30 units. The amount of perioperative blood loss is a significant predictor of the risk of death after hepatic resection.

There is therefore sought a device and method for carrying out resection of tissue which results in a very low amount of bleeding.

### BRIEF DESCRIPTION OF THE DRAWINGS

The invention will now be described further, by way of example only, with reference to the accompanying drawings, in which:

FIG. 1 shows a cross-section of one embodiment of a device;

FIG. 2 shows a perspective view of the device of FIG. 1;

FIG. 3 is a schematic diagram illustrating the arrangement of the electrodes of the device shown in FIGS. 1 and 2;

FIG. 4 shows a perspective view of another embodiment of a device;

FIG. 5 shows a detailed perspective view of a part of the device shown in FIG. 1;

FIG. 6 shows a cross-section of the part of the device shown in FIG. 5;

FIG. 7 shows an example of a switching circuit for use with the embodiment of the device shown in FIG. 4;

FIG. 8 shows an example of a switching circuit for use with the embodiment of the device as shown in FIGS. 1, 2 and 3; and

FIG. 9 shows an example of the active electrodes of a device controlled by the switching circuit of FIG. 8.

### DESCRIPTION OF THE INVENTION

A bipolar surgical resection device and method are described. In the following description, for the purposes of explanation, numerous specific details are set forth in order to provide a thorough understanding of the present invention. It will be apparent, however, to one skilled in the art that the present invention may be practised without these specific details. In other instances, well-known structures and devices are shown in block diagram form to avoid unnecessarily obscuring the present invention.

The needs identified in the foregoing background, and other needs and objects that will become apparent for the following description, are achieved in the present invention, which comprises, in one aspect, a surgical resection device comprising at least two elongate elements for insertion into tissue, each element comprising an electrode capable of operating in a bipolar manner, and an input for receiving a drive signal for driving the electrodes. The electrodes may be arranged in a two-dimensional array. Additionally or alternatively the subsets of the electrodes may be driven in turn.

There is also provided a method of performing surgical tissue resection comprising inserting into tissue a resection device comprising a plurality of elongate electrodes, the electrodes being capable of operating in a bipolar manner and the electrodes being arranged in a two-dimensional array, and driving the electrodes with a drive signal. Additionally or alternatively the method of performing surgical tissue resection may comprise inserting a surgical resection device comprising a plurality of elongate electrodes, the electrodes being capable of operating in a bipolar manner, and driving the electrodes with a drive signal, the device being arranged such that, in use, subsets of the elongate electrodes are driven in turn.

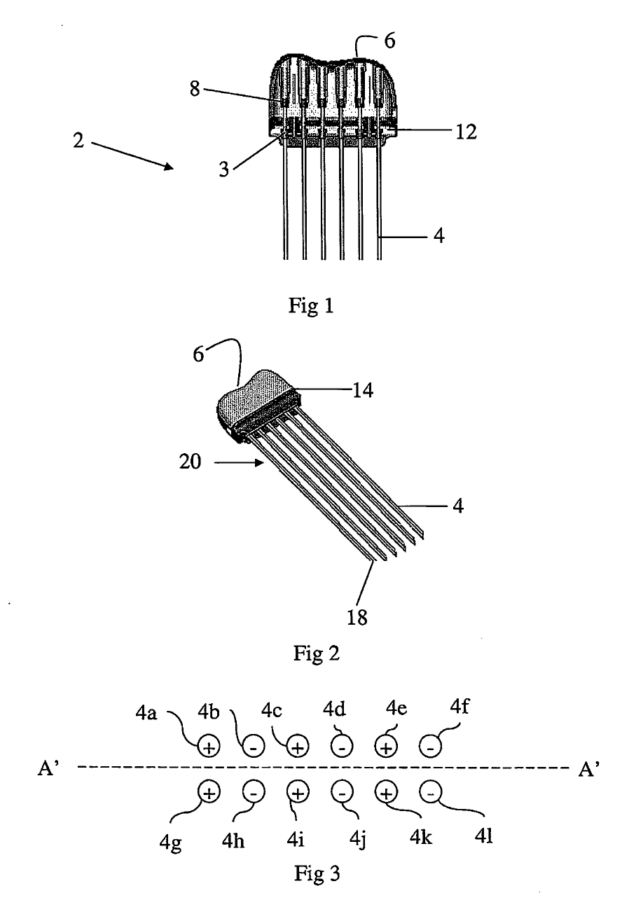

FIGS. 1 and 2 show one embodiment of a surgical organ resection device. The device 2 is a handheld device and comprises a plurality of elongate elements 4, each of which has a portion which operates, in use, as an electrode. In use, the array of elongate elements 4 (also known as needles) are inserted into tissue during surgery. When connected to a radio frequency (RF) generator and driven with appropriate RF energy, tissue in the immediate vicinity of the needles is heated. The tissue heating causes vessel sealing, preventing blood loss during subsequent resection. The device may be designed for single use only.

The device comprises a two dimensional array of elongate elements 4, which array comprises at least two sets of elongate elements, the elongate elements of each set being electrically connected together. In the embodiment shown in FIGS. 1, 2 and 3 the device comprises six sets of elongate elements, each set comprising a pair of elements. However, it will be appreciated by a person skilled in the art that the device may comprise two or more sets of elongate elements arranged in a two-dimensional array, with each set comprising two or more elongate elements 4 electrically connected together. The elongate elements of a set may be permanently electrically connected together or may be electrically switched to be electrically connected together. By being electrically connected together it will be apparent to a person skilled in the art that the elongate elements of a set are of the same electrical polarity.

The device may comprise a plurality of elongate electrodes arranged in a n×m array where n and m are integers greater than or equal to 2. In one embodiment, the m elongate electrodes comprise adjacent elongate electrodes of opposite polarity. Additionally or alternatively, the n elongate electrodes may comprise adjacent elongate electrodes of the same polarity.

FIGS. 1 and 2 shown a device comprising a plurality of elongate electrodes arranged in a n×m array where n is an integer greater than or equal to 2 and m is an integer greater than or equal to 3. In the embodiment shown, n is equal to 2 and m is equal to 6.

In one embodiment, each elongate element 4 comprises a coated needle shaft, an insulation sleeve 5 (e.g. of polyimide or PTFE) and a crimped ferrule. The provision of an insulated sleeve allows for modification of the active electrode heating section of the elongate element. The needles 4 may be made of any appropriate material e.g. stainless steel or copper. They typically have an outer diameter around 1.-5 to 2 mm and are typically 30 mm to 200 mm in length. The distal end is sharpened to a point for ease of insertion. Each individual needle 4 is suitable to withstand typical forces in both push and pull directions. The non-insulated (i.e. active) length of each needle is around 30 mm to 100 mm.

The needles are driven with an RF signal, for instance between 50 kHz and 2 MHz. An RF signal of less than 1 MHz is particularly suitable as conformity with EMC specifications is generally not required at this frequency in many jurisdictions. A suitable RF signal is 400-700 kHz and in particular 480-700 kHz. The typical voltage used will not generally exceed 100 V rms and the current will not generally exceed 3A rms.

The device includes a top shell portion 6 (further detail of which is shown in FIGS. 5 and 6) which includes cavities 60 for receiving a proximal end of an elongate element 4. The top shell is shaped to fit comfortably the palm of the hand of a surgeon. The top shell has an input (not shown) for entry of a cable for driving the electrodes.

A needle pusher 8 and holder 10 are made from the same base component, which is then drilled to suit. These support the needle 4 and clamp a PCB 12, thereby removing any loading on the solder joint. Sealing of the holders 10 using epoxy resin and silicone adhesive minimises risk from fluid ingress.

The PCB 12 is a single sided board with 1 oz Copper track and plated through holes. Two location holes allow the board to be supported, whilst the needle supports clamp the board. Alternatively bus bars may be used rather than a PCB.

A bottom shell 14 incorporates a lip to aid sealing and assembly. Two holes allow for the use of self-tapers to both clamp the assembly together and locate the restraining method used for a push off plate 16.

A cable (not shown) runs from the rear of the device. As the device is intended to be “single use” cable clamping may not be required. A grommet seals the cable and offers some strain relief. The assembly of the top and bottom shells 6, 14 clamps this. At least a portion of the outer surface of the elongate elements 4 (the tissue-contact surface) may have a low coefficient of friction so that the surface of the elongate elements 4 does not stick to the tissue during surgery. To achieve this low coefficient of friction (also referred to as non-stick), the elongate elements 4 may be coated with a material having a low coefficient of friction, such as conductive PTFE (polytetrafluoroethylene), titanium, titanium nitride or the like. Additionally or alternatively, the outer surface of the elongate elements 4 may be highly polished to achieve the low coefficient of friction. The surface energy of the tissue-contact surface should optionally be less than 40 mN/m (milli Newtons per metre) and optionally less than 20 mN/m.

Part of each elongate element may be insulated, in particular the proximal portions of the elongate elements. For instance, non-conductive PTFE may be used on the proximal ends of the elongate elements, as an insulator, with the distal ends being coated in a conductive material having a low coefficient of friction.

The device may be manufactured as follows:
1. The needle assembly 4 is inserted through the PCB 12 and soldered in place. The cable assembly is then attached.
2. The needle holders 10 are then assembled in to the bottom shell 14 with epoxy resin to form a fluid seal and mechanical bond.
3. The needle/PCB assembly is then pushed through the needle holder/bottom shell until only 10 mm is left to bottom out.
4. Silicone adhesive is applied around each needle between the PCB and needle holder and the whole pushed fully home to seal the needles.
5. The needle pushers 8 are then located in to the top shell 6.
6. A bead of adhesive is placed around the sealing lip and this is assembled to the bottom shell assembly. (This minimises fluid ingress)
7. A push-off plate member (if used) is then assembled and attached using two shelf taper screws.

The device operates in a bipolar manner i.e. the current travels from one or more of the electrodes in the device to at least one other of the electrodes of the device. This means that the energy deposition of the device is localised to the area of the device and that it does not travel to a separate electrode provided elsewhere on the patient.

In the embodiment shown in FIGS. 1 and 2, twelve elements 4 are shown arranged as two rows of six elements. Each element in a row is separated from its neighbour by around 4-6 mm. Each row is separated by around 5 to 7.5 mm.

FIG. 3 shows a schematic diagram of the electrodes of the device as shown in FIGS. 1 and 2. Each elongate element 4 comprises an electrode. In the diagram as shown in FIG. 3, the electrodes are arranged in a generally linear formation, with two rows of six electrodes being provided. The device comprises a plurality of electrodes electrically connected together in sets, which, in the embodiment shown, are pairs. Thus the device as shown in FIGS. 1, 2 and 3 comprises six pairs of electrodes, each pair being electrically connected together such that there is a first pair of positive polarity electrodes, followed by a second pair of negative polarity electrodes, followed by a third pair of positive polarity electrodes, and so on. Each alternate electrode acts as the ground return for the active electrode i.e. the electrodes act in a bipolar manner.

Each needle in a row may be connected to the opposite needle in the other row. For the elements in a row, the polarity of one electrode is opposite to that of the adjacent element in the row. The energisation pattern of the electrodes may alter during use. The energisation pattern of the electrodes may be determined by external control apparatus in accordance with the resection required.

The area of resection for a device as shown in FIGS. 1, 2 and 3 is along an axis A′-A′ as shown in FIG. 3. Having a two-dimensional array of elongate elements provides a wider area of resection than a linear array of elongate elements since the volume of tissue between the needles in a set is heated rather than simply the volume between adjacent sets of needles.

The elongate elements 4 may be cooled, in particular with a gas cooling system. Cooled air may be used and a heatsink may also be use, for instance a copper heatsink. Air may be cooled and then forced through the needles 4 with a small pump such as an Interpet Aqua AP2 air pump (not shown).

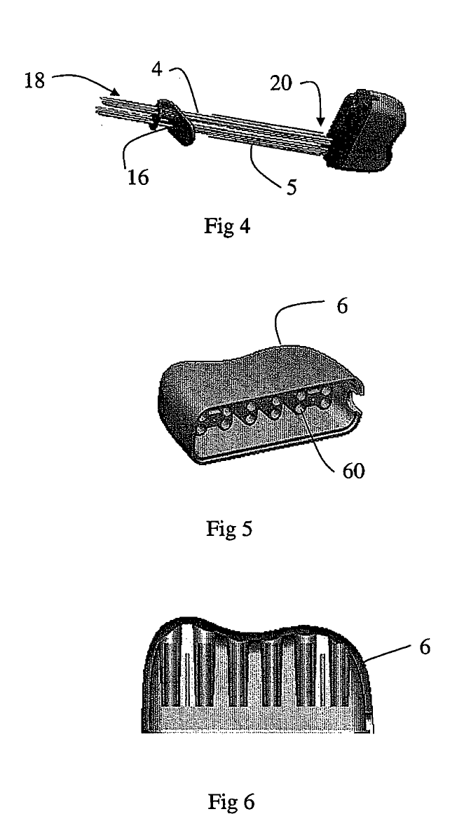

FIG. 4 shows a second embodiment of a surgical organ resection device. In this embodiment, four elongate elements are provided. In addition, a push-off member 16 is provided to maintain the position of the elongate elements with respect to each other. The member 16 may slide along the elongate elements. Before insertion, the member 16 would be adjacent the distal end 18 of the elongate elements 4. As the elongate elements are inserted into the tissue of the organ, the member 16 moves along the elongate elements towards the proximal end 20 of the elongate elements. This assists in maintaining the spatial separation of the elongate elements during insertion and also prevents the tissue being pulled during withdrawal of the elongate elements from the tissue by holding the tissue in position when the elements are withdrawn.

The bottom shell 14 incorporates a lip to aid sealing and assembly. Two holes allow for the use of self-tapers to both clamp the assembly together and locate the restraining method (e.g., a thin cord) used for the member 16.

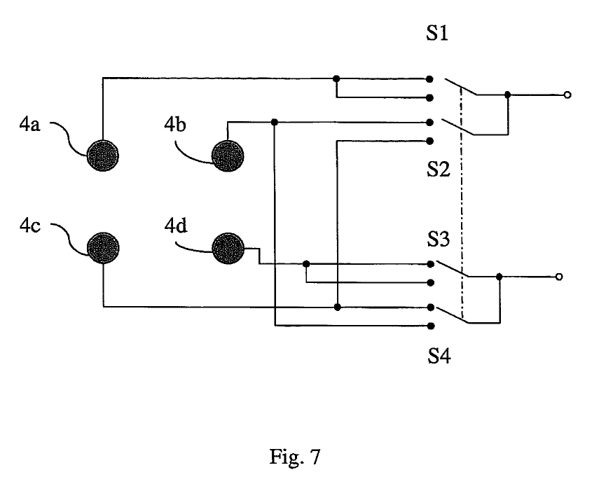

FIG. 7 shows an example of a switching arrangement for a 2 pair device as shown in FIG. 4. The switching arrangement shown allows for the rotation of the heating field. For instance, by suitable operation of switches S1, S2, S3 and S4, the polarity of the electrodes 4a, 4b, 4c and 4d may be altered. Examples of suitable switching patterns may be: electrodes 4a, 4b positive and electrodes 4c, 4d negative; electrodes 4a, 4c positive and electrodes 4b, 4d negative; electrodes 4a, 4d positive and electrodes 4b, 4c negative. This means that the heating field may be rotated to result in a more even heating of the tissue between the electrodes.

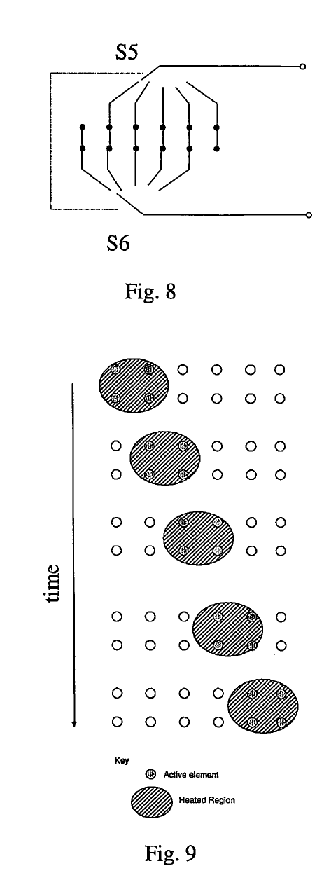

In one embodiment of a surgical organ resection device (for instance as illustrated in the figures (excluding FIGS. 4 and 7)), the device comprises a plurality of elongate electrodes 4 for insertion into organ tissue, the electrodes being capable of operating in a bipolar manner, and an input for receiving a drive signal for driving the electrode, the device being arranged such that, in use, subsets of the elongate electrodes are driven in turn. Thus, in the device as shown in FIGS. 8 and 9, the elongate electrodes are arranged in a two-dimensional array of n×m elongate electrodes, where n and m are integers greater than or equal to 2.

In such an embodiment, in which a device has more than three sets of electrodes (for instance as shown in FIGS. 1, 2 and 3), adjacent pairs of needles may be switched in, so that at a given time only two sets of electrodes are active. This is illustrated in FIG. 8 which shows twelve electrodes 4a-4l and FIG. 9 which shows the active needles of the device as the switching pattern progresses. Thus initially active are a first subset of the needles of the array comprising needles 4a, 4b, 4g and 4h. Once these needles have been active for sufficient time to coagulate the tissue between the active needles, the next subset of needles are made active i.e. needles 4b, 4c, 4h and 4i. Once these needles have been active for sufficient time to coagulate the tissue between the active needles, the next subset of needles are made active i.e. needles 4c, 4d, 4i and 4j, and so on. This means that at a given time only two sets (in this case pairs) of needles are active at a given time even though all needles of the device are inserted into the tissue. Thus, in this embodiment, a six pair device, in essence, operates as a successive series of two pair devices with heating occurring one region at a time, with a step and repeat operation controlled by the switches S5 and S6. Thus removal and re-insertion of the needles of the device is not required which speeds up the operation compared with repeated insertion of a two pair device.

In the device as shown the m elongate electrodes comprise adjacent elongate electrodes of opposite polarity. This may be achieved by a suitable switching arrangement, for instance as shown in FIG. 8. The n elongate electrodes may comprise adjacent elongate electrodes of the same polarity.

In a particular embodiment, the device may comprise a plurality of elongate electrodes arranged in a n by m (n×m) array, and the subsets of elongate electrodes comprise n×p elongate electrodes, where p is an integer less than m.

A resection device has been described that comprises at least two pairs of electrodes arranged in a two dimensional array. However it will be apparent to a person skilled in the art that the device may comprise a two dimensional array of electrodes of n×m electrodes where n and m are integers greater or equal to 2. Thus the device may comprise for instance a 2×2 array, a 2×6 array, a 3×3 array etc.

Such a device as described herein is suitable for use in solid vascular organ surgery e.g. liver, spleen, kidney, pancreas. The device not only seals the blood vessel of the organ but also seals other vessels, such as the bile duct and the pancreatic duct. This prevents bile or pancreatic juices continuing to flow. Preferably in use the device is inserted such that the major axis A′-A′ of the two-dimensional array of the elongate electrodes is orthogonal to the major blood vessels in the tissue to be resected.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

1. A surgical organ resection device comprising:
a plurality of elongate electrodes for insertion into organ tissue, the electrodes being capable of operating in a bipolar manner, and
an input for receiving a drive signal for driving the electrode, the elongate electrodes being arranged in a two-dimensional array.

2. A device according to claim 1 comprising two or more sets of elongate elements arranged in a two-dimensional array, with each set comprising two or more elongate electrodes electrically connected together.

3. A device according to claim 1 or 2 wherein the plurality of elongate electrodes are arranged in a two-dimensional array comprising a plurality of rows of elongate electrodes arranged in a generally linear formation.

4. A device according to claim 3 wherein, in use, an elongate electrode in a first row is of the same polarity as the adjacent elongate electrode in an adjacent row of the two-dimensional array.

5. A device according to any preceding claim wherein, in use, the polarity of one electrode is opposite to the polarity of an adjacent electrode in a given direction in the two-dimensional array.

6. A device according to any preceding claim wherein at least one of the elongate electrodes has a tissue-contact surface that has a surface energy of 40 mN/m or less.

7. A device according to claim 6 wherein the elongate electrode has a coating of PTFE or titanium.

8. A device according to claim 6 wherein the elongate electrode has a highly polished surface.

9. A device according to any preceding claim further comprising means for cooling at least a portion of the elongate electrode using a gas.

10. A device according to claim 9 wherein the gas is air or nitrogen.

11. A device according to any preceding claim comprising a plurality of elongate electrodes arranged in a n×m array where n and m are integers greater than or equal to 2.

12. A device according to claim 11 wherein the m elongate electrodes comprise adjacent elongate electrodes of opposite polarity.

13. A device according to claim 11 or 12 wherein the n elongate electrodes comprise adjacent elongate electrodes of the same polarity.

14. A device according to claim 13 comprising a plurality of elongate electrodes arranged in a n×m array where n is an integer greater than or equal to 2 and m is an integer greater than or equal to 3.

15. A device according to claim 11 comprising four elongate electrodes arranged in a 2×2 array.

16. A surgical organ resection device comprising:
a plurality of elongate electrodes for insertion into organ tissue, the electrodes being capable of operating in a bipolar manner, and
an input for receiving a drive signal for driving the electrode, the device being arranged such that, in use, subsets of the elongate electrodes are driven in turn.

17. A device according to claim 16 wherein the elongate electrodes are arranged in a two-dimensional array.

18. A device according to claim 16 or 17 comprising a plurality of elongate electrodes arranged in a n×m array where n and m are integers greater than or equal to 2.

19. A device according to claim 18 wherein the m elongate electrodes comprise adjacent elongate electrodes of opposite polarity.

20. A device according to claim 19 wherein the n elongate electrodes comprise adjacent elongate electrodes of the same polarity.

21. A device according to claim 20 comprising a plurality of elongate electrodes arranged in a n×m array where n is an integer greater than or equal to 2 and m is an integer greater than or equal to 3.

22. A device according to claim 21 wherein the subsets of elongate electrodes comprise n×p elongate electrodes, where p is an integer les than m.

23. A device according to claim 22 further comprising a switching mechanism for controlling the drive signal.

24. A method of performing surgical tissue resection comprising:
inserting into tissue a resection device comprising a plurality of elongate electrodes, the electrodes being capable of operating in a bipolar manner and the electrodes being arranged in a two-dimensional array, and
driving the electrodes with a drive signal.

25. A method of performing surgical tissue resection comprising:
inserting a surgical resection device comprising a plurality of elongate electrodes, the electrodes being capable of operating in a bipolar manner, and
driving the electrodes with a drive signal, the device being arranged such that, in use, subsets of the elongate electrodes are driven in turn.
